(19) United States
(12) Patent Application Publication
(10) Pub. No.: US 2024/0282433 A1
(43) Pub. Date: Aug. 22, 2024

(54) INFORMATION PROCESSING APPARATUS, RADIATION IMAGING SYSTEM, INFORMATION PROCESSING METHOD, AND NON-TRANSITORY COMPUTER-READABLE STORAGE MEDIUM

(71) Applicant: CANON KABUSHIKI KAISHA, Tokyo (JP)

(72) Inventor: Kento OSUMI, Saitama (JP)

(21) Appl. No.: 18/430,706

(22) Filed: Feb. 2, 2024

(30) Foreign Application Priority Data
Feb. 21, 2023 (JP) 2023-025528

(51) Int. Cl. G16H 30/40; A61B 6/00; A61B 6/46

(52) U.S. Cl. CPC G16H 30/40; A61B 6/465; A61B 6/5294

(57) ABSTRACT

An information processing apparatus includes: a determining unit configured to determine whether to execute second external processing on a radiation image using processing information relating to a first external processing of the radiation image; and a transmitting unit configured to transmit the radiation image to a transmission destination of the second external processing in a case where it is determined to execute the second external processing.

### BACKGROUND OF THE INVENTION

### Field of the Invention

The techniques of the disclosure relate to an information processing apparatus, a radiation imaging system, an information processing method, and a non-transitory computer-readable storage medium.

### Description of the Related Art

In recent years, artificial intelligence (AI) technology has made advancements in the field of medical imaging diagnosis support. A radiation imaging system is installed with image processing for diagnosis support, but by utilizing image processing outside of the radiation imaging system, more support can be obtained that is helpful for the physician. A radiation imaging system can cooperate with an external image processing apparatus (hereinafter also referred to as an external processing apparatus) to transmit and receive an image (radiation image) corresponding to a processing source and a post-processing image (hereinafter also referred to as a processed image) obtained by image processing or the like being executed on the image (radiation image) and to provide an image obtained by processing not installed in the radiation imaging system for a diagnosis. For example, in the configuration described in Japanese Patent Laid-Open No. 2022-26377, an image range determined to be necessary for a diagnosis is specified by an operator, and image data of the specified image range is output to an external apparatus.

Depending on the processing content, there may be a plurality of registered external processing apparatuses cooperating with a radiation imaging system, and during an examination, images may be required to be transmitted to various external processing apparatuses. When an image is transmitted to an external processing apparatus, a mistaken determination or transmission operation by the operator may cause an image to be transmitted to an external processing apparatus that it should not be. This may cause more time than necessary being taken for processing and a delay to the examination time.

The disclosure provides techniques for reducing the operation load of the operator.

### SUMMARY OF THE INVENTION

According to one aspect of the present invention, there is provided an information processing apparatus comprising: a determining unit configured to determine whether to execute second external processing on a radiation image using processing information relating to a first external processing of the radiation image; and a transmitting unit configured to transmit the radiation image to a transmission destination of the second external processing in a case where it is determined to execute the second external processing.

According to another aspect of the present invention, there is provided an information processing apparatus comprising: a determining unit configured to determine one of a plurality of transmission destinations as a transmission destination for executing external processing of a radiation image using processing information relating to the radiation image; and a transmitting unit configured to transmit the radiation image to the determined transmission destination.

According to still another aspect of the present invention, there is provided an information processing method comprising: determining whether to execute second external processing on a radiation image using processing information relating to a first external processing of the radiation image; and transmitting the radiation image to a transmission destination of the second external processing in a case where it is determined to execute the second external processing.

Further features of the present invention will become apparent from the following description of exemplary embodiments (with reference to the attached drawings).

### BRIEF DESCRIPTION OF THE DRAWINGS

FIG. 1 is a diagram illustrating an example configuration of a radiation imaging system according to a first embodiment.

FIG. 2 is a block diagram illustrating the functional configuration of a control unit.

FIG. 3 is a diagram illustrating examples of an imaging method table and an external processing table stored in a storage unit illustrated in FIG. 2.

FIG. 4 is a diagram illustrating an example of an image table for storing captured images, processed images generated by an external processing apparatus, and processing information.

FIG. 5A is a diagram illustrating an example of an examination input screen for a radiation imaging apparatus according to an embodiment.

FIG. 5B is a diagram illustrating an example of an examination input screen for a radiation imaging apparatus according to an embodiment.

FIG. 5C is a diagram illustrating an example of an examination input screen for a radiation imaging apparatus according to an embodiment.

FIG. 6 is a diagram illustrating an example of an imaging screen for a radiation imaging apparatus according to an embodiment.

FIG. 7 is a flowchart illustrating an example of the process of processing to transmit a radiation image to an external processing apparatus when performing imaging.

FIG. 8 is a flowchart illustrating an example of the process of processing to transmit a radiation image to an external processing apparatus when receiving processing information.

FIG. 9 is a flowchart illustrating an example of the process of processing to transmit a radiation image to an external processing apparatus when executing image processing.

FIGS. 10A and 10B are flowcharts illustrating an overall summary of the processing by an information processing apparatus according to an embodiment.

### DESCRIPTION OF THE EMBODIMENTS

Embodiments will be described in detail below with reference to the attached drawings. Note that the invention according to the scope of the claims are not limited by the embodiments described below. A plurality of advantages of the embodiments are given. However, all of the plurality of advantages are not required for the invention. Also, the plurality of advantages may be combined in a discretionary manner. Furthermore, in the attached drawings, the same or equivalent components are denoted with the same reference number, and redundant descriptions will be omitted.

Radiation according to the techniques of the present disclosure includes α-rays, β-rays, and γ-rays, which are beams of particles (including photons) emitted due to radioactive decay, as well as beams with approximately equal or greater energy, such as X-rays, a particle beam, and cosmic rays.

Note that in the embodiments described below, a radiation imaging system is used as an example. However, the modality for imaging a subject may be a magnetic resonance imaging (MRI) apparatus, an X-ray computed tomography (CT) apparatus, a three-dimensional ultrasound imaging apparatus, a photoacoustic tomography apparatus, a positron emission tomography/single photon emission computed tomography (PET/SPECT) apparatus, an optical coherence tomography (OCT) apparatus, or a similar medical imaging apparatus (simply referred to as an imaging apparatus). In other words, a series of processing according to an embodiment can be implemented by a medical information processing system (simply referred to as an information processing system) including various imaging apparatuses and a medical information processing apparatus (simply referred to as an information processing apparatus) and can be applied to not only radiation images but also other medical images. Also, an image relating to the technique of the disclosure includes in its meaning not only a state of being displayed on a display unit but also a state of being stored in a database or a storage unit and a state of being transmitted and received via a network as image data.

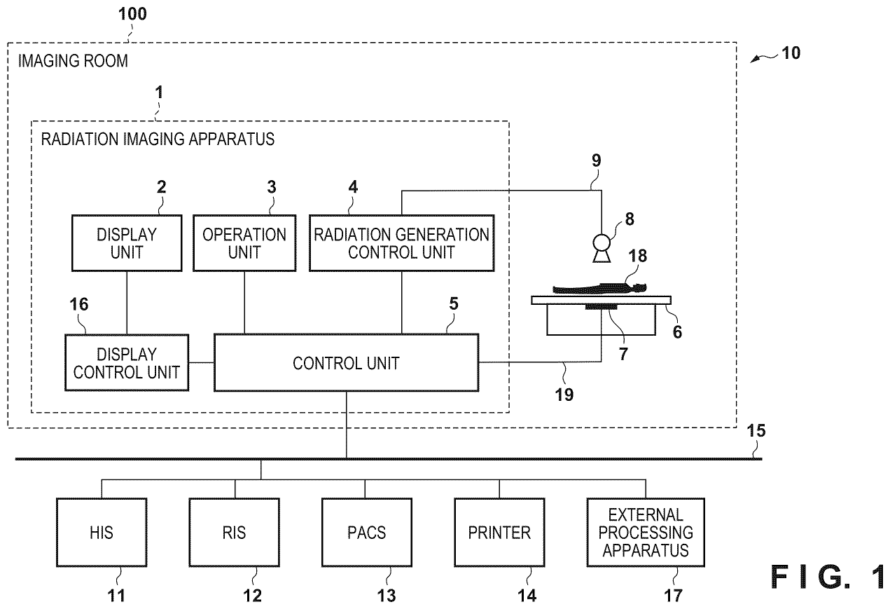

The configuration and operations of a radiation imaging system 10 according to an embodiment will now be described with reference to FIGS. 1 to 6.

### Configuration of Radiation Imaging System

FIG. 1 is a diagram illustrating an example configuration of the radiation imaging system 10 according to a first embodiment. The radiation imaging system 10 includes a radiation imaging apparatus 1 and a radiation detector 7. In the radiation imaging system 10, the radiation imaging apparatus 1 is communicatively connected to a hospital information system (HIS) 11, a radiology information system (RIS) 12, picture archiving and communication systems (PACS) 13, a printer 14, and an external processing apparatus 17 via a network 15. The HIS 11 is a hospital information system for managing the progress of the radiation imaging (examination).

Also, the RIS 12 is a radiology department information system for transferring examination orders to the radiation imaging apparatus 1. The PACS 13 is an image server for managing radiation images. The printer 14 outputs radiation images by printing. The external processing apparatus 17 executes processing on radiation images.

The HIS 11 may include a server for managing accounting information. When radiation imaging is performed, the operator enters an examination instruction using a terminal (input unit) of the HIS 11 and transfers it to the radiology department, which is the request destination. The request information is also referred to as an examination order, and an examination order includes a request source department name, an examination ID, an examination item, patient information (examination subject information) relating to a subject 18 (examination subject), and the like. When the radiology department receives the examination order transmitted from the RIS 12, the imaging information (body part to be imaged information, imaging direction information, procedure information, and the like) relating to the radiation imaging is attached to the examination order as the imaging protocol and transferred to the radiation imaging apparatus 1.

The radiation imaging apparatus 1 performs radiation imaging according to the received examination order. The radiation imaging apparatus 1 obtains the captured radiation image, generates examination information including the radiation image and the examination order in association, and transfers this to the PACS 13 together with the radiation image.

The PACS 13 is a server mainly configured to manage images. Via a high definition monitor connected to the PACS 13, radiation image examination tasks, detailed post-processing, diagnosis tasks, and the like are performed.

Also, the examination information (image ID, date and time of imaging, and the like) for the radiation imaging apparatus 1 is transferred to the HIS 11. The examination information transferred to the HIS 11 is used for examination progress management as well as post-examination accounting processing.

The external processing apparatus 17 is an apparatus that executes image processing and the like on radiation images. Diagnosis support processing or the like using artificial intelligence (AI) technology is executed, and the generated processing information (including a processing result or processed image) is transferred to the radiation imaging apparatus 1, the PACS 13, or the like. The processing information generated by the external processing apparatus 17 is not limited to an image (processed image) and may be information indicating the processing result of various types of image processing such as rejection determination and anomaly detection. The radiation imaging apparatus 1 presents the processing information obtained from the external processing apparatus 17 together with the captured image (original image (radiation image) before being processed at the external processing apparatus) and provides processing not installed in the radiation imaging apparatus 1.

The apparatuses including the radiation imaging apparatus 1, the HIS 11, the RIS 12, the PACS 13, the printer 14, and the external processing apparatus 17 are connected via the network 15 formed by a local area network (LAN) or a wide area network (WAN). Note that each apparatus includes one or more computers. The computer is provided with, for example, a control unit such as a CPU and a storage unit such as read-only memory (ROM), random-access memory (RAM), and the like. Also, the computer may be provided with a communication unit such as a network card, an input/output unit such as a keyboard, a display, or a touch panel, and the like. These components are electrically connected via a bus or the like and are controlled by a main control unit reading out and executing a program stored in a storage unit.

In the radiation imaging system 10, the radiation detector 7 functions as a detector that detects the radiation that passes through the subject 18 and obtains image data (a radiation image) based on the radiation that passes through the subject 18. Specifically, the radiation detector 7 detects the radiation that passes through the subject 18 as a charge corresponding to the transmitted radiation amount. For example, the radiation detector 7 may use a direct conversion sensor that directly converts radiation into a charge using a-Se or the like to convert radiation into a charge or an indirect conversion sensor using a scintillator such as CsI and a photoelectric conversion element such as a-Si.

As illustrated in FIG. 1, the radiation imaging apparatus 1 that performs radiation imaging is installed in an imaging room 100. The radiation detector 7 that captures a radiation image by detecting the radiation that passes through the subject 18 and an imaging platform 6 are installed in the imaging room 100. The radiation detector 7 A/D converts the detected charge to generate image data and store it in a non-illustrated storage unit. The radiation detector 7 can provide the image information (image ID, imaging date and time, and image data transfer state) to the image data (radiation image) and transfer it together with the image data to the radiation imaging apparatus 1. The radiation detector 7 can be installed in the imaging platform 6, for example.

### Configuration of Radiation Imaging Apparatus 1

The radiation imaging apparatus 1 includes a display unit 2 that displays radiation images and various types of information, an operating unit 3 operated by an operator, a radiation generation control unit 4, a display control unit 16, and a control unit 5 that controls these components.

The display unit 2 is implemented by a liquid crystal display or the like and displays various types of information to the operator (for example, an imaging technician, physician, or the like). The operating unit 3 is constituted by a mouse, an operation button, or the like and is for the operator to input various types of instruction to the components. Note that the display unit 2 and the operating unit 3 may be integrally implemented as a touch panel.

The radiation generation control unit 4 is connected to a radiation generator 8 via a cable 9, and the radiation generation control unit 4 sets the radiation imaging condition for the radiation generator 8 and controls the emission of radiation from the radiation generator 8.

The radiation generator 8 functions as a radiation source that generates radiation. The radiation generator 8, for example, is implemented by a radiation tube and emits radiation toward the subject 18 (for example, a specific body part of the subject). On the emission surface of the radiation generator 8, a non-illustrated diaphragm for blocking radiation is provided. The radiation generator 8 emits radiation in a desired emission area. The radiation generation control unit 4 can adjust the emission area of the radiation from the radiation generator 8 by controlling the diaphragm for blocking radiation.

The control unit 5 is connected to the radiation detector 7 via a wireless LAN. Image information (image ID, imaging date and time, and image data transfer state), image data, control signals, and the like are exchanged between the control unit 5 and the radiation detector 7. The control unit 5 generates a control signal in accordance with a set imaging condition and controls the radiation detector 7 on the basis of the control signal. The image data (radiation image) and image information stored in the radiation detector 7 via radiation imaging are output (transferred) to the control unit 5 via the wireless LAN. Note that the control unit 5 and the radiation detector 7 may be connected via a cable 19.

### Functional Configuration of Control Unit 5

The control unit 5 of the radiation imaging apparatus 1 executes image processing on the image data (radiation image) obtained from the radiation detector 7 and generates an image. The control unit 5, for example, includes a central processing unit (CPU) or a dedicated or general-purpose processor, for example. The processor implements the various types of functions described below by executing various types of programs stored in a storage unit 23 in the control unit 5. The control unit 5 may be constituted by a field programmable gate array (FPGA), an application specific integrated circuit (ASIC), or a similar piece of hardware. The control unit 5 may be constituted by a single processor or by a combination of a plurality of independent processors. The various types of functions described below can be implemented by these types of hardware. Also, the control unit 5 can implement various types of functions via a combination of software processing by a processor and a program and hardware processing.

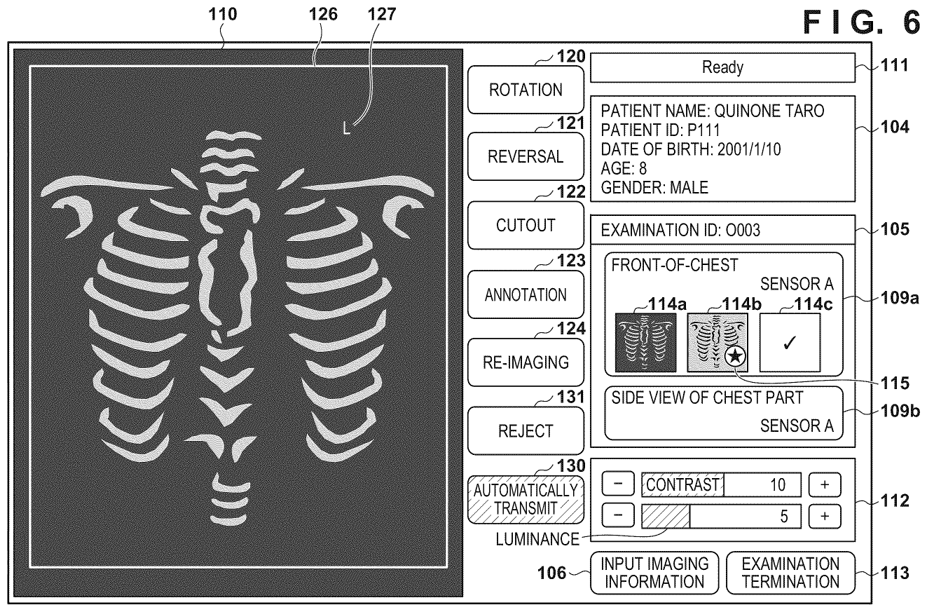

The control unit 5 controls the radiation detector 7 and outputs the radiation image obtained from the radiation detector 7 to the display unit 2 and outputs a graphical user interface (GUI) to the display unit 2. The control unit 5, together with the display control unit 16, performs display control to display, on the display unit 2 (for example, first regions 109a and 109b in FIG. 6), a thumbnail image of the radiation image (captured image) obtained from the radiation detector 7 and a thumbnail image of the processed image obtained by executing image processing and the like on the radiation image using the external processing apparatus 17. Also, the control unit 5 functions as a reception unit for receiving image edit instruction relating to the radiation image (captured image) displayed on the display unit 2. The control unit 5 can perform image editing of the radiation image (captured image) by cropping, annotation, and the like on the basis of image edit instructions from the operator.

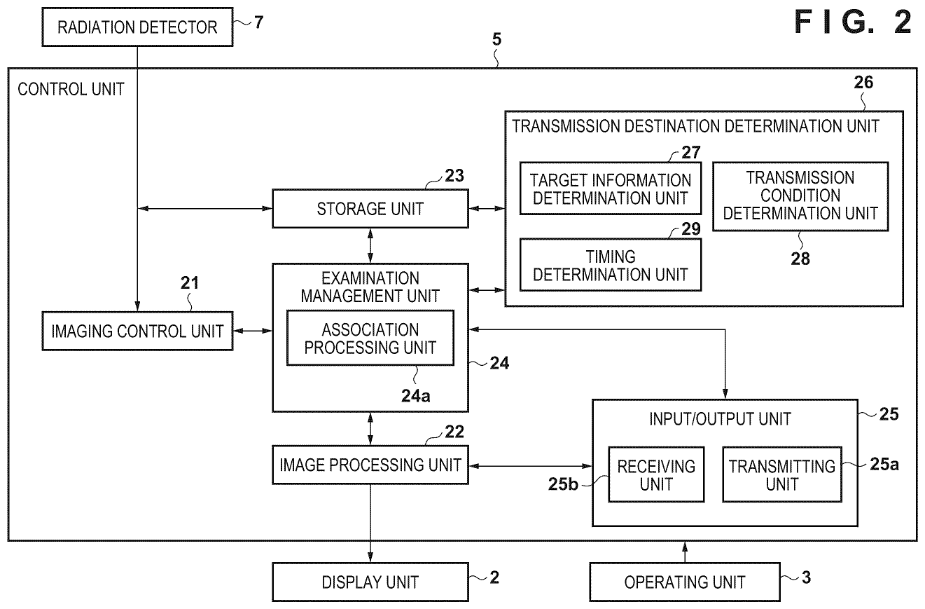

FIG. 2 is a block diagram illustrating the functional configuration of the control unit 5. The control unit 5 includes an imaging control unit 21 for performing imaging control of the radiation detector 7, an image processing unit 22 for executing image processing of the radiation image obtained via imaging, and the storage unit 23 for storing the radiation image output from the radiation detector 7, examination orders, imaging protocols, imaging methods, and similar types of information.

Also, the control unit 5 includes an examination management unit 24 for managing the examination information including the radiation image and examination order in association and an input/output unit 25 for inputting or outputting the generated image objects and processing information to the outside.

The storage unit 23 stores examination information managed by the examination management unit 24, imaging protocols, imaging methods, radiation images output from the radiation detector 7, various types of information required for examination management, and registration information of the external processing apparatus 17 registered with the radiation imaging apparatus 1. The control unit 5 also includes a transmission destination determination unit 26 that, in a case where processing information is received from the external processing apparatus 17, automatically determines the transmission destination on the basis of the received processing information for when transmitting to the next external processing apparatus.

The transmission destination determination unit 26 searches and retrieves (obtains) various types of setting conditions for executing processing for transmission to the external processing apparatus 17 from an external processing table 3B registered in the storage unit 23. The transmission destination determination unit 26 includes, as functional configurations, a target information determination unit 27, a transmission condition determination unit 28, and a timing determination unit 29.

The transmission condition determination unit 28 performs condition determination for transmitting transmission target information (captured image) to the external processing apparatus 17, that is, the processing request destination (transmission destination), for each registered external processing apparatus 17 (A to D).

The target information determination unit 27 determines whether or not the information to be applied to the condition determination of the transmission condition is predetermined information (for example, a captured image or processing information).

The timing determination unit 29 determines whether or not the timing for applying the determination target (determination target information) to the condition determination of the transmission condition is predetermined timing (for example, the imaging time, the time of receiving processing information, the time of executing image processing, or the like).

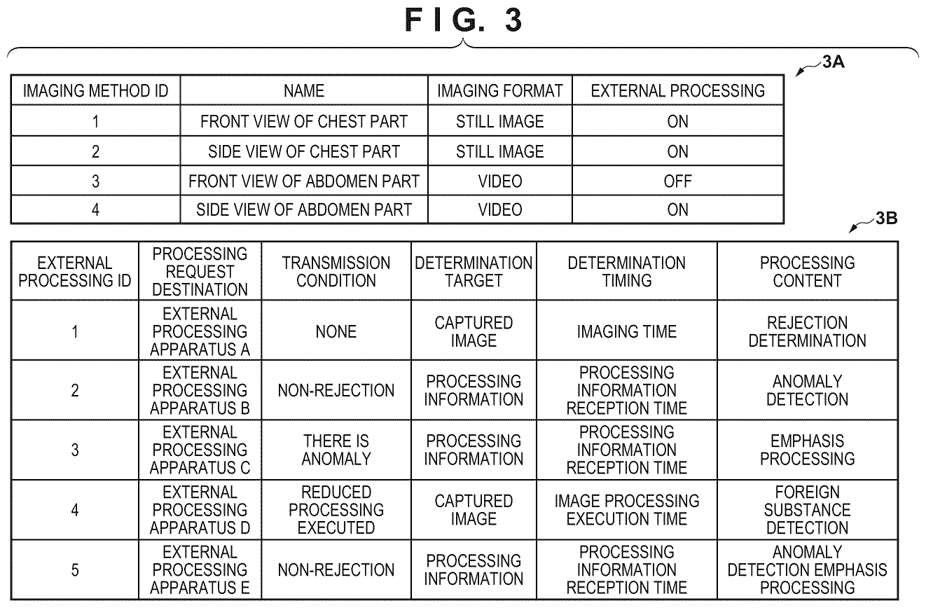

FIG. 3 is a diagram illustrating examples of an imaging method table and an external processing table stored in the storage unit 23 illustrated in FIG. 2. An imaging method table 3A illustrated in FIG. 3 is a table for storing, for each imaging method, an imaging method ID provided for identifying (specifying) the imaging method, a name, imaging format (still image, video), setting values for whether external processing is enabled (ON) or disabled (OFF), and the like. Here, the external processing setting being enabled (ON) means that external processing is set to be executed on radiation images, and the external processing setting being disabled (OFF) means that external processing is set to not be executed on radiation images. For example, in the case of the imaging method ID1 with a still image being captured of a front view of the chest part, the external processing setting is enabled (ON) as indicated. In a similar manner, in the case of the imaging method ID3 with a video being captured of a front view of the abdomen part, the external processing setting is disabled (OFF) as indicated.

The external processing table 3B is a table including, for each external processing ID provided to identify external processing, a processing request destination (transmission destination), a transmission condition, a determination target, a determination timing, and processing contents in association. In the external processing table 3B, a plurality of external processing apparatuses A to D are registered as the request destinations (transmission destinations) of external processing.

In the external processing table 3B, a transmission condition is a condition for transmitting transmission target information (captured image) to the external processing apparatus 17 which is the processing request destination. For the transmission condition, a different transmission condition is set for each external processing apparatus 17 which is a processing request destination. A setting of “no condition” where no transmission condition is set is also included in the transmission condition settings.

When a transmission condition is met, the control unit 5 controls the input/output unit 25 (a transmitting unit 25a) and causes it to transmit the transmission target information (captured image) to the predetermined external processing apparatus 17 (for example, a first external processing apparatus (external processing apparatus A) or a fourth external processing apparatus (external processing apparatus D)). Here, the transmission target captured image (radiation image) is an original image prior to processing by the external processing apparatus 17.

In the external processing table 3B, the determination target (determination target information) indicates the information to be applied to the condition determination of the transmission condition. The determination target includes a captured image and processing information.

For example, when applying a captured image as a determination target to the condition determination of the transmission condition and a predetermined transmission condition is met, the control unit 5 controls the input/output unit 25 (transmitting unit 25a) and causes it to transmit the transmission target information (captured image) to the predetermined external processing apparatus 17 (for example, the first external processing apparatus (external processing apparatus A) or the fourth external processing apparatus (external processing apparatus D)).

Alternatively, in a case where the determination target is processing information, for example, when applying processing information (first processing information) generated by the first external processing apparatus to the condition determination of the transmission condition as a determination target and a predetermined transmission condition is met, the control unit 5 controls the input/output unit 25 (transmitting unit 25a) and causes it to transmit the transmission target information (captured image) to the predetermined external processing apparatus 17. In this case, the control unit 5 controls the input/output unit 25 (transmitting unit 25a) and causes it to transmit the transmission target information (captured image) to a different external processing apparatus 17 (for example, a second external processing apparatus (external processing apparatus B) or a third external processing apparatus C) to the external processing apparatus 17 (first external processing apparatus).

In the external processing table 3B, the determination timing indicates the timing at which the determination target (determination target information) is applied to the condition determination of the transmission condition. The determination timing is, for example, the time when the captured image is obtained (imaging time), the time when image processing is executed on the captured image (image processing execution time), the time when the processing information is received (processing information reception time), or the like.

Also, in the external processing table 3B, the processing content indicates the processing content of each external processing apparatus. For example, for the external processing ID1, the captured image is transmitted to the external processing apparatus A with the determination timing corresponding to the imaging time when the captured image is obtained. Rejection determination is set as the processing content for the external processing apparatus A, and the external processing apparatus A performs rejection determination on the captured image and transmits the rejection determination result as the processing information to the information processing apparatus which is the transmission source of the captured image.

Note that in the external processing table 3B, the transmission condition, the processing content, the processing request destination (transmission destination), the determination target, and the determination timing can be set as desired. For example, regarding the transmission condition for non-rejection, a plurality of processing request destinations (transmission destinations) can be set. The transmission condition determination unit 28 can select the transmission destination from among the plurality of processing request destinations (for example, the external processing apparatus B and the external processing apparatus E) on the basis of the setting of the external processing apparatus that can execute the processing content required for the external processing. The transmission condition determination unit 28 uses the processing information relating to the radiation image to determine which of the plurality of transmission destinations to use as the transmission destination for executing external processing of the radiation image. For example, in a case where the processing content required as external processing is only anomaly detection, the transmission condition determination unit 28 selects the external processing apparatus B as the transmission destination. In a case where the processing content required as external processing is anomaly detection and emphasis processing, the transmission condition determination unit 28 selects the external processing apparatus E as the transmission destination. Also, regarding the plurality of external processing apparatuses, if there is a case in which the set processing content is the same, the transmission condition determination unit 28 can compare the processing loads of the external processing apparatuses on the basis of the information (for example, number of pieces of image data, image data amount, and the like) of transmitted radiation images and select the external processing apparatus with the lower processing load as the transmission destination. In this manner, external processing congestion can be reduced, and the external processing result can be obtained earlier.

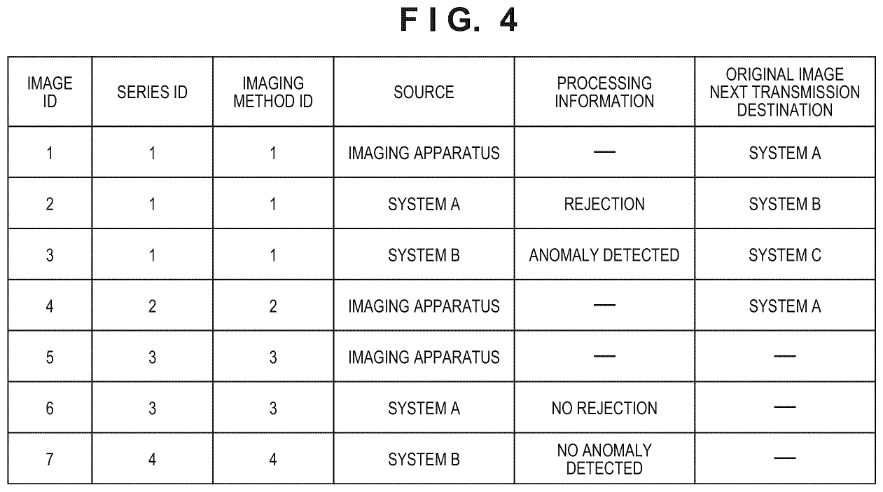

FIG. 4 is a diagram illustrating an example of an image table for storing captured images, processed images generated by an external processing apparatus, and image information of processed images stored in the storage unit 23 illustrated in FIG. 2. An image ID provided to each image (or processing information), a series ID, an imaging method ID, an image (or processing information) generation source, processing information obtained via processing by the external processing apparatus, and the next transmission destination for the captured image are stored in the image table.

Here, the series ID is information for associating together the captured image obtained by the radiation imaging apparatus 1 via the imaging method specified by the imaging method ID and processing information (processed image) obtained via processing by the external processing apparatus 17. For example, regarding the imaging method ID1 (for example, front view of chest part, still image), the captured image with image ID1, the processing information (processed image) with image ID2, and the processing information (processed image) with image ID3 that share the same series ID1 are associated together.

Also, in the image table, the next transmission destination is the next transmission destination (next transmission destination of original image) of the captured image (radiation image) for determination of whether to transmit or not on the basis of the processing information. The next transmission destination, for example, is the next transmission destination (second external processing apparatus: system B) of the captured image for determination of whether to transmit or not on the basis of the processing information obtained via processing by the external processing apparatus 17 (system A) as the first external processing apparatus.

In a case where the imaging information (radiation image) is to be transmitted to a different external processing apparatus, the transmitting unit 25a transmits the captured image (radiation image) obtained by the radiation imaging apparatus 1 as the original image to the external processing apparatus 17 (system A: first external processing apparatus).

A receiving unit 25b receives the processing information generated from the captured image (radiation image) via the processing executed by the first external processing apparatus. The transmission condition determination unit 28 determines whether or not the processing information received by the receiving unit 25b corresponds to the transmission condition for transmitting the captured image (radiation image) to at least one second external processing apparatus different from the first external processing apparatus. The transmission destination determination unit 26 determines the transmission destination via the determination result of the transmission condition determination unit 28.

The imaging control unit 21 and the operating unit 3 function as a setting unit for setting the condition for the transmission condition determination unit 28 to execute determination processing relating to the various types of table settings in FIGS. 3 and 4. The imaging control unit 21 and the operating unit 3 (setting unit) set at least one of: when imaging for a radiation image is performed, when processing information is received, and when image processing is executed on the radiation image as the timing for performing determination, for example, and store this in the various types of tables (FIGS. 3 and 4) in the storage unit 23. Also, the imaging control unit 21 and the operating unit 3 (setting unit) set at least one of the radiation image and the processing information as the type of information to be the target of determination and stores this in the various types of tables (FIGS. 3 and 4) in the storage unit 23. Also, the imaging control unit 21 and the operating unit 3 (setting unit) set at least one of: the result of rejection determination, the result of anomaly detection, the result of emphasis processing, and the result of foreign substance detection as the content of the processing executed on the information to be the target of determination and store this in the various types of tables (FIGS. 3 and 4) in the storage unit 23.

The image of the image ID1 is the captured image (radiation image) obtained by the radiation imaging apparatus 1 by executing the imaging method specified by the imaging method ID1 (for example, front view of chest part, still image). Processing information (processed image) of the image ID2 and processing information (processed image) of the image ID3 are generated with the captured image (radiation image) as the original image. Here, the processing information (processed image) of the image ID2 is the processing information (processed image) obtained by processing at the external processing apparatus 17 (system A) being executed, and the processing information (processed image) of the image ID3 is the processing information (processed image) obtained by processing being executed at the external processing apparatus 17 (system B).

In a similar manner, the image of the image ID4 is the captured image (radiation image) obtained by the radiation imaging apparatus 1 by executing the imaging method specified by the imaging method ID2 (for example, side view of chest part, still image). Also, the image of the image ID5 is the captured image (radiation image) obtained by the radiation imaging apparatus 1 by executing the imaging method specified by the imaging method ID3 (for example, front view of abdomen part, video), and the processing information (processed image) of the image ID6 is generated with the captured image (radiation image) of the image ID5 as the original image. The processing information (processed image) of the image ID6 is the processing information (processed image) obtained by processing at the external processing apparatus 17 (system A) being executed.

The examination management unit 24 manages the imaging protocol associated with the examination order and has a defined imaging method, imaging condition, image processing condition, and the like. For example, in a case where examination information is generated at the radiation imaging apparatus 1, the examination management unit 24 can associate together the subject information input via the operating unit 3 and the imaging protocol and generate new examination information. In a case where an examination is requested from the RIS 12, the examination management unit 24 uses the identification information of the imaging protocol associated with the received examination order and obtains the imaging protocol stored in the storage unit 23. Here, the imaging method ID that may be included in the imaging protocol may be used as the identification information of the imaging protocol to obtain the imaging protocol. The examination management unit 24 associates the obtained imaging protocol with the examination order and generates new examination information. The examination management unit 24 stores the newly generated examination information in the storage unit 23. Also, an association processing unit 24a of the examination management unit 24 uses supplementary information attached to the processing information (for example, processed image) and identification information of the captured image (radiation image) to associate together the captured image and the processing information. The examination management unit 24 stores the generated information in the storage unit 23. Here, the supplementary information includes identification information (image ID) for identifying the captured image (radiation image) and information indicating the processing content to be executed by the external processing apparatus 17. Also, the identification information of the captured image (radiation image) is information (image ID) for identifying (specifying) a radiation image.

The imaging control unit 21 transmits, to the radiation detector 7, a transfer request signal for requesting a transfer of the radiation image held by the radiation detector 7 and receives (obtains) the radiation image from the radiation detector 7. The imaging control unit 21 associates the received (obtained) radiation image with radiation detector information relating to the radiation detector 7 and manages them. Also, the imaging control unit 21 associates together the radiation image obtained from the radiation detector 7 and the examination information and imaging protocol managed by the examination management unit 24.

The image processing unit 22 uses the imaging protocol, the examination information, and the radiation image associated together by the imaging control unit 21 to execute image processing on the radiation image. Here, the radiation image obtained via image processing by the image processing unit 22 is displayed on the display unit 2. Alternatively, the radiation image obtained via image processing by the image processing unit 22 is transmitted to the outside by the transmitting unit 25a of the input/output unit 25. The image processing unit 22 executes image processing to adjust the image itself in terms of brightness and contrast, for example. Also, the image processing unit 22 can execute editing processing such as cropping and annotation on the post-adjustment radiation image.

The input/output unit 25 includes the transmitting unit 25a for controlling transmission processing and the receiving unit 25b for controlling reception processing. The receiving unit 25b of the input/output unit 25 receives examination orders from the RIS 12, accepts processed image input from the external processing apparatus 17, and the like. Also, the transmitting unit 25a of the input/output unit 25 outputs the radiation image to an external apparatus such as the PACS 13, the printer 14, the external processing apparatus 17, and the transmitting unit 25a of the input/output unit 25 also outputs the examination information (image ID, imaging date and time, and the like) to the HIS 11.

The example described above has been of the functional configuration of the radiation imaging system 10 and the radiation imaging apparatus 1 according to an embodiment. Note that the configurations illustrated in FIGS. 1 and 2 are examples and can be changed as appropriate. For example, in FIG. 1, the radiation imaging apparatus 1 is connected to the various types of apparatuses via the network 15, but the radiation imaging apparatus 1 does not necessarily need to be connected to the various types of apparatuses via the network 15. For example, a diagnosis image may be output to a portable storage medium such as a DVD and input to the various types of apparatuses via the portable storage medium. Also, the network 15 may have a wired configuration, or a part of the network 15 may be configured by a wireless signal transmission path.

### Imaging Processing

Next, a process will be described of the processing executed when a radiation image is captured according to the flow of examination by the radiation imaging system 10 illustrated in FIG. 1. First, the subject information (patient information) and examination information are input into the radiation imaging apparatus 1 via an examination request form or an examination request from the RIS 12. Here, the subject information includes, as information for specifying the subject, the subject name (patient name), subject identification information (subject ID or patient ID), and the like, and the examination information includes examination identification information (examination ID), imaging information specifying the content of the imaging to be performed on the subject, and the like.

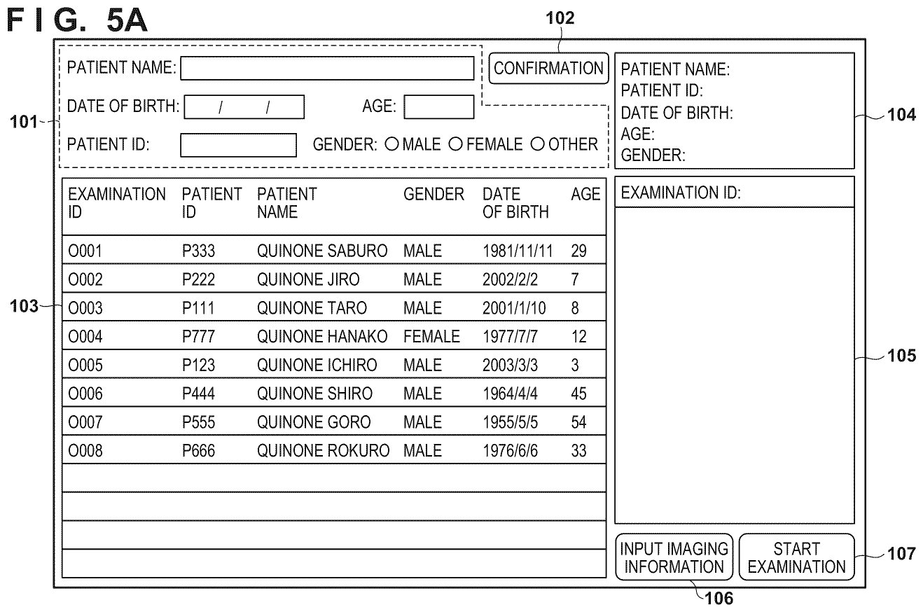

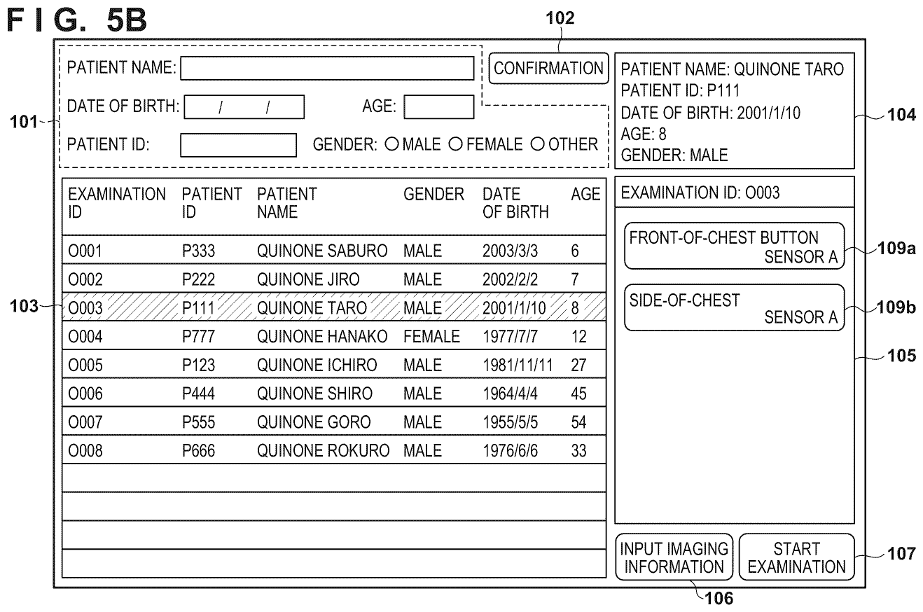

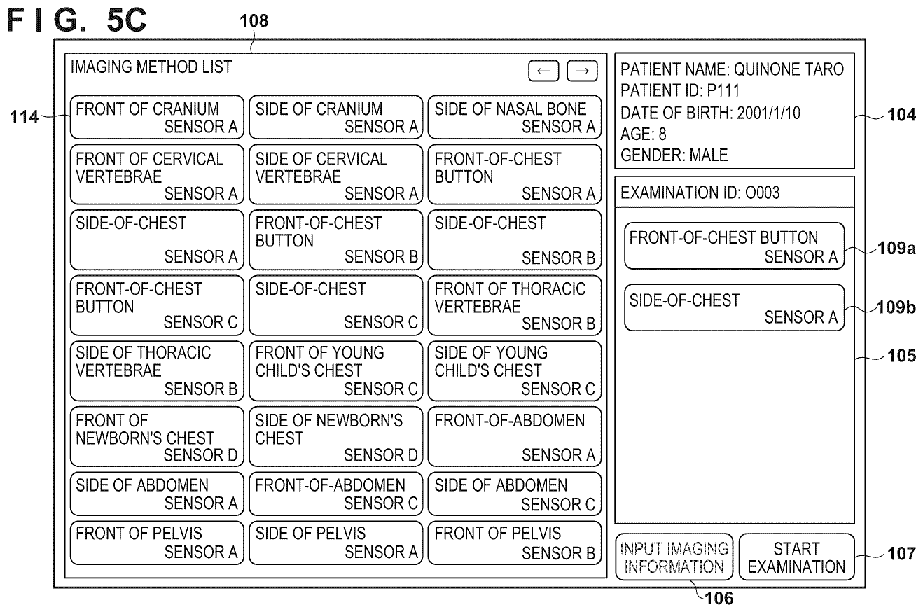

FIGS. 5A to 5C are diagrams of examples of examination input screens for the radiation imaging apparatus 1 of an embodiment, which are also examples of imaging screens in the display unit 2 of the radiation imaging apparatus 1 according to an embodiment.

The radiation imaging apparatus 1 displays a new examination input screen such as that illustrated in FIG. 5A on the display unit 2 via display control by the display control unit 16. As illustrated in FIG. 5A, the configuration of the new examination input screen includes an input region 101 for subject information, a confirmation button 102 for subject information, and a request examination list 103. Also, the configuration of the new examination input screen includes a display region 104 for subject information, a display region 105 for imaging information, an imaging information input button 106, and an examination start button 107.

The display control unit 16 perform display control to display the examination information (subject information and examination information) received by the RIS 12 in the request examination list 103 in the form of a list. When one of the examinations is selected from the display of the request examination list 103, as illustrated in FIG. 5B, in the display region 104 for subject information, the subject information (subject ID (patient ID)), subject name (patient name), date of birth, and the like) corresponding to the selected examination is displayed. Also, in the display region 105 for imaging information, the examination ID corresponding to the selected examination is displayed, and in the region below, the imaging information corresponding to the examination ID is displayed. As described above, the display control unit 16 can display the imaging information based on the information received from the RIS 12.

In the case of the example in FIG. 5B, the display control unit 16 performs display control to display an imaging method button 109 (for example, a front view of chest part button 109a and a side view of chest part button 109b) corresponding to the imaging information in the display region 105 for imaging information. When the imaging information input button 106 is pressed, as illustrated in FIG. 5C, the display control unit 16 performs display control to display an imaging condition input region 108 on the display unit 2. The imaging method can be further added by a select button 114 of the plurality of imaging methods (imaging conditions) displayed in the imaging condition input region 108 being selected. The display control unit 16 performs display control to display the added imaging method next to the front view of chest part button 109a and the side view of chest part button 109b in the display region 105 for imaging information. Each imaging method (imaging condition) is associated with an imaging method ID (imaging condition ID).

The operator presses the examination start button 107 after checking the subject information (display region 104 for subject information) and the imaging information (display region 105 for imaging information). In this manner, the examination to be performed is confirmed. When the examination start button 107 is pressed, the display control unit 16 of the radiation imaging apparatus 1 displays an imaging screen such as that illustrated in FIG. 6 on the display unit 2. The imaging screen illustrated in FIG. 6 is a screen used when imaging.

The imaging screen illustrated in FIG. 6 basically includes similar display regions as in the new examination input screen described using FIGS. 5A to 5C. As illustrated in FIG. 6, examples of the newly added display regions include an image display region 110, a message region 111, an image processing settings region 112, and an examination end button 113.

When the imaging screen is displayed, the display control unit 16 performs display control to display the imaging method button 109 (for example, the front view of chest part button 109a) located highest up in the display region 105 for imaging information in a default selected state. Accordingly, the control unit 5 of the radiation imaging apparatus 1 transmits, to the radiation generation control unit 4, the imaging condition (tube voltage, tube current, irradiation time, and the like) set according to the selected imaging method button (imaging method). Then, the control unit 5 controls the radiation detector 7 according to the set imaging condition and prepares for imaging.

When preparations are complete, the control unit 5 of the radiation imaging apparatus 1 transitions the state of the radiation detector 7 to an imaging ready state. At this time, the display control unit 16 performs display control to display a “ready message” indicating the imaging ready state in the message region 111.

Next, the operator checks the imaging method and performs setting for imaging and positioning of the subject. When this series of imaging preparations are complete, the operator checks that the state is the imaging ready state by referencing the message region 111 before pressing the radiation irradiation switch (not illustrated). Then, the control unit 5 of the radiation imaging apparatus 1 causes the radiation generator 8 to emit radiation toward the subject 18 (specific body part of the patient) and causes the radiation detector 7 to detect the radiation that passes through the subject 18. In this manner, radiation image imaging is performed.

When the imaging is complete, the control unit 5 of the radiation imaging apparatus 1 obtains a captured image from the radiation detector 7 and performs image processing on the obtained captured image on the basis of a predetermined image processing condition. The predetermined image processing condition is set in advance according to the selected imaging method button (imaging method).

Also, the display control unit 16 of the radiation imaging apparatus 1 performs display control to display the captured image obtained via image processing in the image display region 110. Also, the display control unit 16 generates thumbnail images 114a to 114c to be displayed in the imaging method button 109. The display control unit 16 displays, in the first region 109a of the display unit 2, the thumbnail image 114a of the radiation image, the first thumbnail image 114b indicating the processing information relating to first external processing, and the second thumbnail image 114c indicating state of the radiation image after being transmitted to the transmission destination of second external processing. In a case where the operator checks the captured image displayed in the image display region 110 and wishes to change the contrast or the like of the captured image, the operator operates the buttons for contrast, brightness, and the like provided in the image processing settings region 112.

In a similar manner, in a case where the operator wishes to change the cropping region of the output image, the operator operates a cropping button 122, a cropping frame button 126, and the like to specify the desired cropping region. To add characters corresponding to diagnosis information, the operator operates an annotation button 123 or the like and overlays characters such as that indicated by annotation 127 on the image. In a case where the orientation of the image is not appropriate for diagnosis, the operator performs geometric transformation using a rotate button 120, an invert button 121, and the like. To perform re-imaging, the operator uses a re-imaging button 124 and performs radiation imaging again. As described above, the operator can operate the various types of buttons to perform additional image editing on the captured image displayed in the image display region 110.

In a case where the image processing of the external processing apparatus 17 is used, the operator presses an automatic transmission button 130 via the operating unit 3. When the automatic transmission button 130 is pressed, the pressed state is maintained until the examination is complete or until the automatic transmission button 130 is pressed again, and the automatic transmission setting applied to the external processing apparatus is maintained. For the imaging method, whether or not external processing is enabled and the external processing request destination, transmission condition, determination target, determination timing, and external processing content are set in advance (for example, the imaging method table 3A and the external processing table 3B in FIG. 3). In a state in which the automatic transmission setting is applied, at the set determination timing, if the determination target is determined to meet the transmission condition, the radiation image corresponding to the processing target can be automatically transmitted to all of the registered external processing apparatus 17 which meet the transmission condition. In other words, in a case where a plurality of the external processing apparatuses 17 are provided and a plurality of transmission destinations meet the transmission condition, the image is transmitted to the plurality of external processing apparatuses 17 all at once, and in a case where no transmission destinations meet the transmission condition, nothing is transmitted to the external processing apparatuses 17. Alternatively, the control unit 5 can set whether or not a medical image (radiation image) can be transmitted to the external processing apparatus 17.

The transmission condition is a condition for transmitting the captured image or the processing information to the external processing apparatus 17 which is the processing request destination. The determination target indicates the target information for determining whether or not the transmission condition is met, and the determination timing indicates the timing of when to execute the processing to determine whether or not the target information meets the transmission condition.

The display control unit 16, for example, performs display control to display an icon image indicating the state of the performed external processing request (sent processing request) in the imaging method button 109 as illustrated by the thumbnail image 114c.

The examination order received by the RIS 12 includes the imaging information. Regarding the imaging information, by setting external processing to enabled, generation of an image obtained by the external processing apparatus 17 executing predetermined external processing can be requested from the HIS 11 and the RIS 12.

When processing information obtained via predetermined external processing is obtained from the external processing apparatus 17, the control unit 5 uses the captured image ID (hereinafter, also referred to as the image ID), to which the processing information is attached, to associate together the captured image and the processing information. In other words, the association processing unit 24a of the examination management unit 24 uses the supplementary information of the processing information as the association information for associating together the captured image (original image) and the processing information to search and retrieve the original image of the processing from the storage unit 23 (for example, the image table in FIG. 4) and specify the captured image (original image). The association processing unit 24a of the examination management unit 24 associates together the retrieved captured image (original image) and the processing information. Information relating to the processing information such as the captured image ID may be obtained via communication other than reception of processing information. In other words, the association processing unit 24a of the examination management unit 24 may use association information obtained from the external processing apparatus 17 via communication with a different timing to the communication performed when obtaining the processing information from the external processing apparatus 17 to search and retrieve the captured image (original image) corresponding to the processing source of the processing information from the storage unit 23 and associate together the retrieved captured image (original image) and the processing information. The processing information associated with the captured image (original image) is displayed as a thumbnail image in the same imaging method button 109a (first region) as the captured image.

In other words, the control unit 5, together with the display control unit 16, perform display control to cause the display unit 2 (for example, in the first regions 109a and 109b in FIG. 6) to display a thumbnail image of the original image (radiation image (captured image)) and a thumbnail image of the processing information obtained from the external processing apparatus 17 based on their association.

For example, the control unit 5 and the display control unit 16 displays, in the first region (109a, 109b, and the like) of the display unit 2, the thumbnail image 114a of the radiation image, the first thumbnail image 114b indicating the processing information relating to first external processing, and the second thumbnail image 114c indicating state of the radiation image after being transmitted to the transmission destination of second external processing. In a case where processing information relating to the second external processing is received, the control unit 5 and the display control unit 16 change the display state of the second thumbnail image 114c to the display state of the first thumbnail image 114b. In a case where processing information of a sent processing request is received, the control unit 5 and the display control unit 16 perform display control to change the display state of the thumbnail image 114c indicating the state that an external processing request has been performed (processing request has been sent) to a display state such as that in the thumbnail image 114b indicating the received processing information.

An icon 115 is an icon for that enables the thumbnail image 114a of the radiation image and the thumbnail image 114b of the processing information (processed image) to be identified. By changing how the icon 115 is displayed (type, display color, or the like), the external processing apparatus that executed the external processing and the processing content can be displayed in an identifiable manner. For example, regarding the display color, the display control unit 16 can change the display color for the first external processing (for example, external processing apparatus A, rejection determination) to a first display color and the display color for the second external processing (for example, external processing apparatus B, anomaly determination) to a second display color. Also, regarding type, the display control unit 16 can change the type of display such as the icon 115 to a different shape such as a circle or a rectangle.

On the thumbnail image, the icon 115 is displayed for identifying between the thumbnail image being processing information generated at the external processing apparatus 17, the external processing apparatus that executed the external processing, and the external processing content. In other words, the control unit 5 and the display control unit 16 can display the thumbnail image 114b of the processing information in a different display form to the thumbnail image 114a of the original image before processing to allow processing information obtained via processing by the external processing apparatus 17 to be identifiable.

When the operator presses (selects) the thumbnail image via the operating unit 3, the control unit 5 and the display control unit 16 cause the processing information of the pressed (selected) thumbnail image to be displayed in the image display region 110. Regarding the processing information, in a case where re-processing by the radiation imaging apparatus 1 is set to not required, the control unit 5 and the display control unit 16 can disable the rotate button 120, the invert button 121, the cropping button 122, the annotation button 123, the image processing settings region 112, and the like to not accept operations from the user interface for image processing and editing. Alternatively, regarding processing information, in a case where re-processing by the radiation imaging apparatus 1 is set to not required and there is an instruction for image editing of the processing information, the control unit 5 and the display control unit 16 can cause a warning indicating that processing is restricted for the image to be displayed on the display unit 2.

When the imaging method button 109b is specified for the next imaging, the control unit 5 prepares for imaging by controlling the radiation detector 7 according to the imaging condition. The control unit 5 transmits, to the radiation generation control unit 4, the imaging condition (tube voltage, tube current, irradiation time, and the like) set according to the selected imaging method button 109b (imaging method). Then, the control unit 5 controls the radiation detector 7 according to the set imaging condition and prepares for imaging. When preparations are complete, the control unit 5 transitions the state of the radiation detector 7 to an imaging-ready state. At this time, the display control unit 16 performs display control to display a “ready message” indicating the imaging ready state in the message region 111. With the imaging method button 109b specified and the radiation emission performed, the image confirmation of the imaging of the imaging method button 109a may be considered to be complete.

The operator repeats the process described above and performs imaging of all of the imaging methods (imaging conditions) in the display region 105 for imaging information. When all of the imaging is complete, the operator presses the examination end button 113. In this manner, the series of examinations ends. The radiation imaging apparatus 1, using the control unit 5, provides the examination information, imaging condition, and the like as the supplementary information, and then outputs the image object to the PACS 13, the printer 14, the storage unit 23 of the apparatus, or the like.

Here, regarding the processed image, for example, in the imaging method table 3A in FIG. 3, whether to output to the image management server (PACS 13) can be set in advance for each imaging method, and the control unit 5 (input/output unit 25) outputs only the processed images of the imaging methods set as enabled for output processing to an image management server to the image management server. In other words, the control unit 5 (input/output unit 25) controls the output of the processed image to the image management server on the basis of the setting of whether or not output of a processed image to an image management server is enabled. Also, the control unit 5 (input/output unit 25) transmits examination information notifying that examination by the radiation imaging apparatus 1 has ended to the HIS 11. The identification information of the processed image received up until the examination end is included in the examination information as an object in the examination. Also, the display control unit 16 of the radiation imaging apparatus 1 causes the new examination input screen to again be displayed on the display unit 2 (FIG. 5A).

### Transmission Flow Directly After Imaging

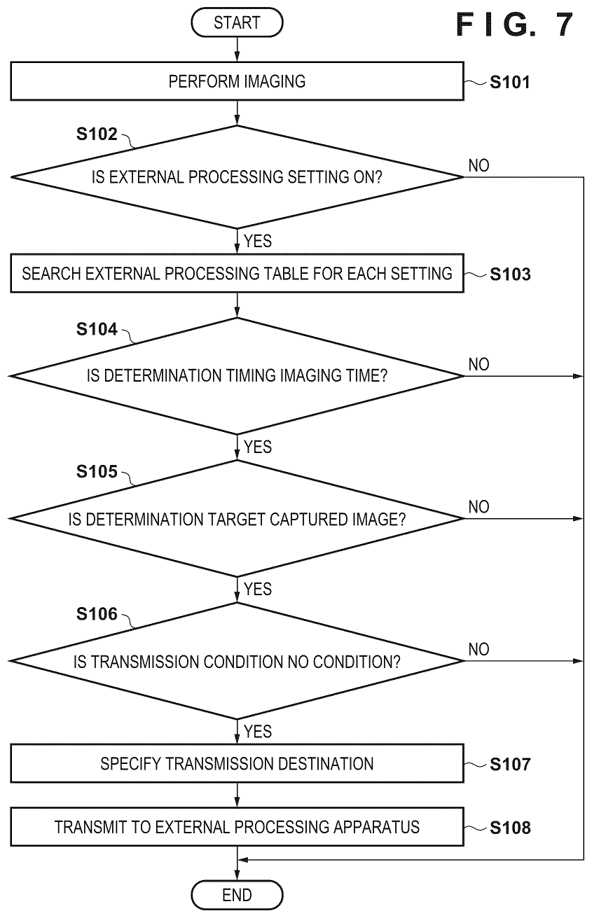

FIG. 7 is a flowchart illustrating an example of the process for processing to transmit the captured image (radiation image) to the external processing apparatus 17 when performing imaging as an information processing method by the radiation imaging apparatus 1 according to an embodiment.

In step S101, the operator performs imaging according to a preset imaging method. For the imaging method of the captured image (radiation image), whether or not external processing is enabled is set (ON/OFF) for each imaging method as illustrated in the imaging method table 3A in FIG. 3.

In step S102, the control unit 5 references the imaging method table 3A (FIG. 3) and determines whether the setting for external processing for the imaging method of the captured radiation image is set to enabled (ON) or disabled (OFF). In the determination processing of step S102, in a case where the external processing setting is disabled (NO in step S102), the control unit 5 ends the processing of the flowchart in FIG. 7. In the determination processing of step S102, in a case where the external processing setting is enabled (YES in step S102), the control unit 5 proceeds the processing to step S103.

In step S103, the transmission destination determination unit 26 of the control unit 5 searches and retrieves (obtains) various types of setting conditions for executing processing for transmission to the external processing apparatus 17 from the external processing table 3B in FIG. 3 already registered in the storage unit 23.

In step S104, the timing determination unit 29 of the control unit 5 determines whether or not the timing for applying the determination target (determination target information) to the condition determination of the transmission condition is predetermined timing. The timing determination unit 29 determines whether, of the obtained setting conditions, the determination timing setting is the imaging time. In a case where the determination timing setting is not the imaging time (NO in step S104), the timing determination unit 29 of the control unit 5 ends the processing of the flowchart in FIG. 7.

In the determination processing of step S104, in a case where the determination processing is the imaging time (YES in step S104), the timing determination unit 29 of the control unit 5 proceeds the processing to step S105.

In step S105, the target information determination unit 27 of the control unit 5 determines whether or not the information to be applied to the condition determination of the transmission condition is predetermined information. The target information determination unit 27 determines whether, of the obtained setting conditions, the determination target setting is the captured image (radiation image). In a case where the determination target setting is not the captured image (NO in step S105), the target information determination unit 27 of the control unit 5 ends the processing of the flowchart in FIG. 7.

In the determination processing of step S105, in a case where the determination target setting is the captured image (YES in step S105), the target information determination unit 27 of the control unit 5 proceeds the processing to step S106.

In step S106, the transmission condition determination unit 28 of the control unit 5 performs condition determination for transmitting transmission target information (captured image) to the external processing apparatus 17, that is, the processing request destination, for each registered external processing apparatus 17 (A to D). The transmission condition determination unit 28 determines, of the obtained setting conditions, the setting for the transmission condition for transmitting the captured image to the external processing apparatus 17 which is the processing request destination. In a case where the transmission condition setting is not “no condition” where no transmission condition is set (NO in step S106), the transmission condition determination unit 28 of the control unit 5 ends the processing of the flowchart in FIG. 7.

In the determination processing of step S106, in a case where the transmission condition setting is “no condition” (YES in step S106), the transmission condition determination unit 28 of the control unit 5 proceeds the processing to step S107.

In step S107, the transmission destination determination unit 26 of the control unit 5 specifies the transmission destination of the captured image on the basis of the processing request destination setting obtained from the external processing table 3B in FIG. 3.

In step S108, the control unit 5 controls the input/output unit 25 (transmitting unit 25a), causing it to transmit the captured image (radiation image) to the external processing apparatus 17 specified as the transmission destination, and ends the processing of the flowchart in FIG. 7.

In the state with the automatic transmission button 130 pressed, the image object (captured image) displayed in the image display region 110 (FIG. 6) is output to the predetermined external processing apparatus 17 according to the transmission condition set for each external processing apparatus 17 (A to D).

### Transmission Flow When Receiving Processing Information

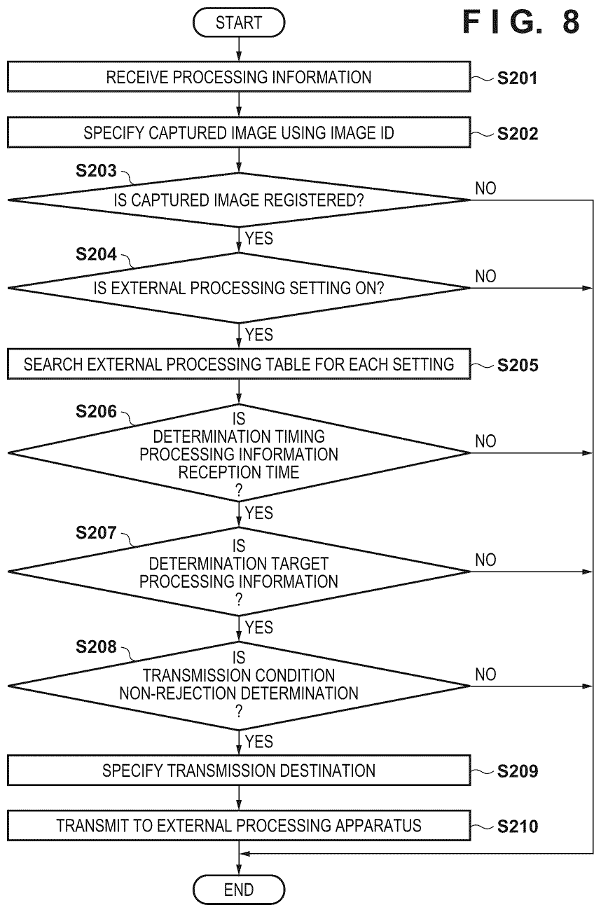

FIG. 8 is a flowchart illustrating an example of the process for processing to transmit the captured image (radiation image) to the external processing apparatus 17 when receiving processing information as an information processing method by the radiation imaging apparatus 1 according to an embodiment.

When imaging is performed in the state with the automatic transmission button 130 pressed, the image object (captured image) displayed in the image display region 110 (FIG. 6) is output to the predetermined external processing apparatus 17 according to the transmission condition set for each external processing apparatus 17 (A to D) following the transmission flow described using FIG. 7.

In step S201, the input/output unit 25 (receiving unit 25b) receives (obtains) the processing information obtained via the predetermined external processing from the external processing apparatus 17.

In step S202, the control unit 5 specifies the captured image using the image ID. The processing information received from the external processing apparatus 17 is attached with an image ID, and the control unit 5 uses the image ID attached to the processing information to associate together the captured image and the processing information. In other words, the examination management unit 24 of the control unit 5 is provided with the association processing unit 24a that associates together the captured image (original image) and the processing information. The association processing unit 24a of the examination management unit 24 uses the image ID attached to the processing information as the association information for associating together the captured image (original image) and the processing information to search and retrieve the original image of the processing from the storage unit 23 (for example, the image table in FIG. 4) and specify the captured image (original image). The association processing unit 24a of the examination management unit 24 associates together the processing information, the radiation image as the original image of the processing information, and the transmission destination (next transmission destination of the original image) on the basis of the image table in FIG. 4.

In step S203, the control unit 5 determines whether or not the captured image (original image) is registered in the image table by searching using the image ID. In a case where the captured image is not registered in the image table (NO in step S203), the control unit 5 ends the processing of the flowchart in FIG. 8. In a case where the captured image is registered in the determination processing of step S203 (YES in step S203), the control unit 5 proceeds the processing to step S204.

In step S204, the control unit 5 references the imaging method table 3A (FIG. 3) and determines whether the setting for external processing for captured image is set to enabled (ON) or disabled (OFF). In a case where the external processing setting for the captured image is disabled (NO in step S204), the control unit 5 ends the processing of the flowchart in FIG. 8.

In the determination processing of step S204, in a case where the external processing setting for the captured image is enabled (YES in step S204), the control unit 5 proceeds the processing to step S205.

In step S205, the control unit 5 searches and retrieves (obtains) various types of setting conditions for executing processing for transmission to the external processing apparatus 17 from the external processing table 3B registered in the storage unit 23.

In step S206, the timing determination unit 29 of the control unit 5 determines whether or not the timing for applying the determination target (determination target information) to the condition determination of the transmission condition is predetermined timing. The timing determination unit 29 determines whether, of the obtained setting conditions, the determination timing setting is the processing information reception time. In a case where the determination timing setting is not the processing information reception time (NO in step S206), the timing determination unit 29 of the control unit 5 ends the processing of the flowchart in FIG. 8.

In the determination processing of step S206, in a case where the determination processing is the processing information reception time (YES in step S206), the timing determination unit 29 of the control unit 5 proceeds the processing to step S207.

In step S207, the target information determination unit 27 of the control unit 5 determines whether or not the information to be applied to the condition determination of the transmission condition is predetermined information. The target information determination unit 27 determines whether, of the obtained setting conditions, the determination target setting is the processing information. In a case where the determination target setting is not the processing information (NO in step S207), the target information determination unit 27 of the control unit 5 ends the processing of the flowchart in FIG. 8.

In the determination processing of step S207, in a case where the determination target setting is the processing information (YES in step S207), the target information determination unit 27 of the control unit 5 proceeds the processing to step S208.

In step S208, the transmission condition determination unit 28 of the control unit 5 performs condition determination for transmitting transmission target information (captured image) to the external processing apparatus 17, that is, the processing request destination, for each registered external processing apparatus 17 (A to D). The transmission condition determination unit 28 determines, of the obtained setting conditions, the setting for the transmission condition for transmitting the captured image to the external processing apparatus 17 which is the processing request destination. The transmission condition determination unit 28 determines whether or not the transmission condition setting is a rejection (non-rejection determination). In a case where the transmission condition setting is not a non-rejection determination (NO in step S208), the transmission condition determination unit 28 of the control unit 5 ends the processing of the flowchart in FIG. 8.

In the determination processing of step S208, in a case where the transmission condition setting is a non-rejection determination (YES in step S208), the transmission condition determination unit 28 of the control unit 5 proceeds the processing to step S209.

In step S209, the transmission destination determination unit 26 of the control unit 5 specifies the transmission destination of the processing information on the basis of the processing request destination setting obtained from the external processing table 3B in FIG. 3.

In step S210, the control unit 5 controls the input/output unit 25 (transmitting unit 25a), causing it to transmit the captured image (radiation image) to the external processing apparatus 17 specified as the transmission destination, and ends the processing of the flowchart in FIG. 8.

### Transmission Flow When Executing Image Processing

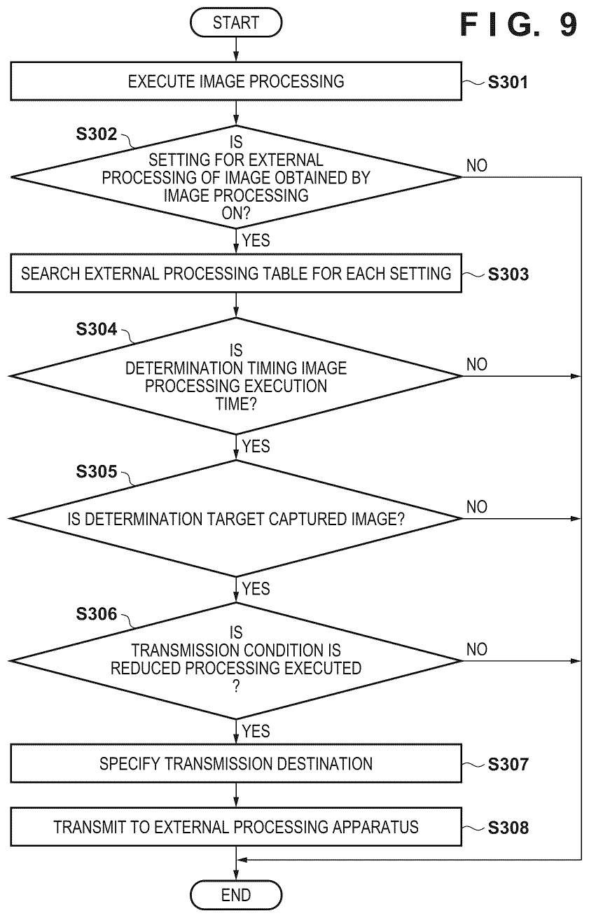

FIG. 9 is a flowchart illustrating an example of the process for processing to transmit the captured image (radiation image) to the external processing apparatus 17 when executing image processing as an information processing method by the radiation imaging apparatus 1 according to an embodiment.

In step S301, the image processing unit 22 executes image processing of the radiation image obtained via imaging.

In step S302, the control unit 5 references the imaging method table 3A (FIG. 3) and determines whether the setting for external processing for the imaging method of the captured image (radiation image) obtained via image processing is set to enabled (ON) or disabled (OFF). In the determination processing of step S302, in a case where the external processing setting is disabled (NO in step S302), the control unit 5 ends the processing of the flowchart in FIG. 9. In the determination processing of step S302, in a case where the external processing setting is enabled (YES in step S302), the control unit 5 proceeds the processing to step S303.

In step S303, the transmission destination determination unit 26 of the control unit 5 searches and retrieves (obtains) various types of setting conditions for executing processing for transmission to the external processing apparatus 17 from the external processing table 3B in FIG. 3 already registered in the storage unit 23.

In step S304, the timing determination unit 29 of the control unit 5 determines whether or not the timing for applying the determination target (determination target information) to the condition determination of the transmission condition is predetermined timing. The timing determination unit 29 determines whether, of the obtained setting conditions, the determination timing setting is the image processing execution time. In a case where the determination timing setting is not the image processing execution time (NO in step S304), the timing determination unit 29 of the control unit 5 ends the processing of the flowchart in FIG. 9.

In the determination processing of step S304, in a case where the determination processing is the image processing execution time (YES in step S304), the timing determination unit 29 of the control unit 5 proceeds the processing to step S305.

In step S305, the target information determination unit 27 of the control unit 5 determines whether or not the information to be applied to the condition determination of the transmission condition is predetermined information. The target information determination unit 27 determines whether, of the obtained setting conditions, the determination target setting is the captured image (radiation image). In a case where the determination target setting is not the captured image (NO in step S305), the target information determination unit 27 of the control unit 5 ends the processing of the flowchart in FIG. 9.

In the determination processing of step S305, in a case where the determination target setting is the captured image (YES in step S305), the target information determination unit 27 of the control unit 5 proceeds the processing to step S306.

In step S306, the transmission condition determination unit 28 of the control unit 5 performs condition determination for transmitting transmission target information (captured image) to the external processing apparatus 17, that is, the processing request destination, for each registered external processing apparatus 17 (A to D). The transmission condition determination unit 28 determines, of the obtained setting conditions, the setting for the transmission condition for transmitting the captured image to the external processing apparatus 17 which is the processing request destination. In a case where the transmission condition setting is not “reduced processing executed” (NO in step S306), the transmission condition determination unit 28 of the control unit 5 ends the processing of the flowchart in FIG. 9.

In a case where the transmission condition is reduced processing executed (YES in step S306), in step S307, the transmission destination is confirmed, then in step S308, a radiation image is transmitted to the confirmed external processing apparatus, and then the flowchart in FIG. 9 ends.

In the determination processing of step S306, in a case where the transmission condition setting is “reduced processing executed” (YES in step S306), the transmission condition determination unit 28 of the control unit 5 proceeds the processing to step S307.

In step S307, the transmission destination determination unit 26 of the control unit 5 specifies the transmission destination of the captured image on the basis of the processing request destination setting obtained from the external processing table 3B in FIG. 3.

In step S308, the control unit 5 controls the input/output unit 25 (transmitting unit 25a), causing it to transmit the captured image (radiation image) to the external processing apparatus 17 specified as the transmission destination, and ends the processing of the flowchart in FIG. 9.

### Overall Summary of Processing by Information Processing Apparatus

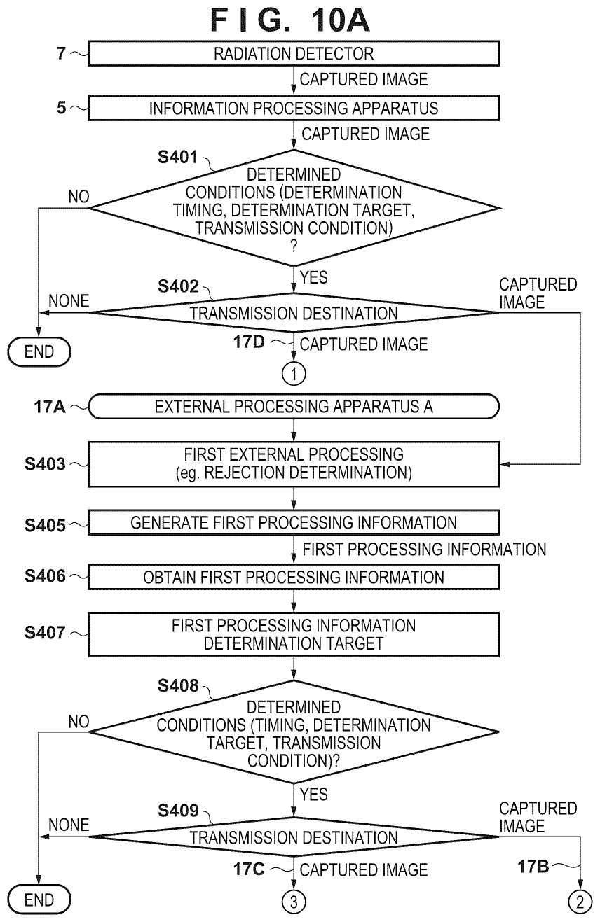

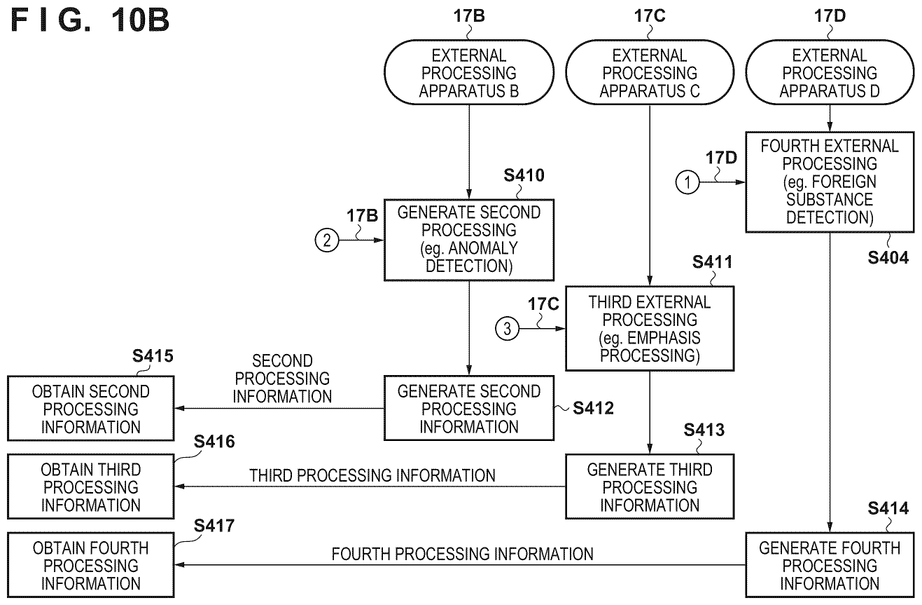

FIGS. 10A and 10B are flowcharts illustrating an overall summary of the processing by the information processing apparatus (control unit 5) according to an embodiment. The condition determination in step S401 is the compilation of the determination processing in FIG. 7 and FIG. 9, and step S408 is a compilation of the determination processing in FIG. 8. The transmission condition determination unit 28 of the control unit 5 determines whether the radiation image (captured image) or the processing information matches the condition relating to the timing for performing determination, the type of information corresponding to the target for determination, and the content of the processing to be executed on the information corresponding to the target and specifies the transmission destination (step S402, step S409) in a case where the radiation image (captured image) or the processing information matches the condition (YES in step S401, YES in step S408).

In step S401, when the radiation image is obtained or when the image processing is executed on the radiation image, the transmission condition determination unit 28 determines whether to execute external processing (for example, first external processing) on the radiation image. In a case where it is determined to execute external processing (first external processing) on the radiation image, the transmission condition determination unit 28 proceeds the processing to step S402. In a case where the first external processing (for example, rejection determination) is set as the transmission destination (step S402, 17A), the transmitting unit 25a transmits the radiation image (captured image) to the first external processing transmission destination (external processing apparatus 17A). In a case where the fourth external processing (for example, foreign substance detection) is set as the transmission destination (step S402, 17D), the transmitting unit 25a transmits the radiation image (captured image) to the fourth external processing transmission destination (external processing apparatus 17D). Here, the transmitting unit 25a transmits a still image or a video to the transmission destination as the radiation image. In a case where a transmission destination is not set for the transmission destination determination in step S402 by the transmission condition determination unit 28, the transmitting unit 25a does not transmit the radiation image.

In step S403, the external processing apparatus A performs rejection determination on the radiation image (captured image), then in step S405, the external processing apparatus A generates the first processing information as the rejection determination result and sends this to the information processing apparatus.

In a similar manner, in step S404, the external processing apparatus D performs foreign substance detection on the radiation image (captured image), then in step S414, the external processing apparatus D generates the fourth processing information as the foreign substance detection result and sends this to the information processing apparatus.

In step S406, the receiving unit 25b receives (obtains) the processing information (first processing information) transmitted from the external processing apparatus 17A. The transmission condition determination unit 28 sets the processing information (first processing information) received (obtained) by the receiving unit 25b as the determination target (step S407).

In step S408, when the processing information is obtained, the transmission condition determination unit 28 determines whether to execute the external processing (for example, the second external processing) different from the first external processing.

In a case where it is determined to execute the external processing (for example, the second external processing) via a determination using the processing information, the transmission condition determination unit 28 determines whether or not there is a transmission destination setting for executing the second external processing. In a case where a transmission destination is set, the transmitting unit 25a transmits the radiation image to the second external processing transmission destination. In a case where the second external processing (for example, anomaly detection) is set as the transmission destination (step S409, 17B), the transmitting unit 25a transmits the radiation image (captured image) to the second external processing transmission destination (external processing apparatus 17B). In a case where the third external processing (for example, emphasis processing) is set as the transmission destination (step S402, 17C), the transmitting unit 25a transmits the radiation image (captured image) to the third external processing transmission destination (external processing apparatus 17C). In a case where a transmission destination is not set for the transmission destination determination in step S409 by the transmission condition determination unit 28, the transmitting unit 25a does not transmit the radiation image.

In step S410, the external processing apparatus B performs anomaly detection on the radiation image (captured image), then in step S412, the external processing apparatus B generates the second processing information as the anomaly detection result and sends this to the information processing apparatus.

In a similar manner, in step S411, the external processing apparatus C performs emphasis processing on the radiation image (captured image), then in step S413, the external processing apparatus C generates the third processing information as the emphasis processing result and sends this to the information processing apparatus.

In step S415, the receiving unit 25b receives (obtains) the processing information (second processing information) transmitted from the external processing apparatus 17B, and in step S416, the receiving unit 25b receives (obtains) the processing information (third processing information) transmitted from the external processing apparatus 17C, and in step S417, the receiving unit 25b receives (obtains) the processing information (fourth processing information) transmitted from the external processing apparatus 17D. The receiving unit 25b receives, as the processing information, the information of the processing result for the radiation image or the processed image generated via the external processing (first external processing to fourth external processing).

According to the techniques of the disclosure, the operating burden on the operator can be reduced.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-025528, filed Feb. 21, 2023 which is hereby incorporated by reference herein in its entirety.

What is claimed is:

1. An information processing apparatus comprising:
a determining unit configured to determine whether to execute second external processing on a radiation image using processing information relating to a first external processing of the radiation image; and
a transmitting unit configured to transmit the radiation image to a transmission destination of the second external processing in a case where it is determined to execute the second external processing.

2. The information processing apparatus according to claim 1, wherein the determining unit determines whether the processing information matches a condition relating to a timing for performing the determination, a type of information corresponding to a target for the determination, and content of processing to be executed on the information corresponding to the target and specifies a transmission destination in a case where the condition is matched.

3. The information processing apparatus according to claim 1, wherein the determining unit determines whether to execute the second external processing when the processing information is obtained.

4. The information processing apparatus according to claim 1, wherein external processing executed on the radiation image using an external processing apparatus which is the transmission destination includes at least one of rejection determination, anomaly detection, emphasis processing, and foreign substance detection.

5. The information processing apparatus according to claim 1, wherein the determining unit determines whether to execute the first external processing on the radiation image, and
the transmitting unit transmits the radiation image to a transmission destination of the first external processing in a case where it is determined to execute the first external processing.

6. The information processing apparatus according to claim 5, wherein the determining unit determines whether to execute the first external processing on the radiation image when the radiation image is obtained or when image processing is executed on the radiation image.

7. The information processing apparatus according to claim 1, wherein the determining unit determines whether or not a setting for executing external processing on the radiation image is set,
in a case where a setting for executing the external processing is set, obtains a condition for the determination set for executing transmission processing for transmitting the radiation image to an external processing apparatus, and specifies the transmission destination in a case where the condition for the determination is matched, and
the transmitting unit
transmits the radiation image to the specified transmission destination, and
does not transmit the radiation image in a case where the transmission destination is not set.

8. The information processing apparatus according to claim 1, wherein the determining unit determines whether or not there is a setting for a transmission destination for executing the second external processing in a case where a determination using the processing information determines to execute the second external processing, and
the transmitting unit
transmits the radiation image to a transmission destination of the second external processing in a case where the transmission destination is set, and
does not transmit the radiation image in a case where the transmission destination is not set.

9. The information processing apparatus according to claim 1, wherein the transmitting unit transmits a still image or video to the transmission destination as the radiation image.

10. The information processing apparatus according to claim 1, further comprising a setting unit configured to set a condition for the determining unit to perform the determination,
wherein the setting unit
sets at least one of when imaging for the radiation image is performed, when the processing information is received, and when image processing is executed on the radiation image as timing for performing the determination,
sets at least one of the radiation image and the processing information as a type of information corresponding to a target for the determination, and
sets at least one of a result of rejection determination, a result of anomaly detection, a result of emphasis processing, and a result of foreign substance detection as content of processing executed on information corresponding to the target for the determination.

11. The information processing apparatus according to claim 1, further comprising a receiving unit configured to receive the processing information,
wherein, with the radiation image being an original image, the receiving unit receives, as the processing information, information of a processing result for the radiation image or a processed image generated via the first external processing.

12. The information processing apparatus according to claim 1 further comprising an associating unit configured to associate together the processing information, the radiation image, and the transmission destination,
wherein the determining unit specifies the radiation image associated with the processing information and the transmission destination.

13. The information processing apparatus according to claim 1 further comprising a display control unit,
wherein the display control unit displays, in a first region of a display unit, a thumbnail image of the radiation image, a first thumbnail image indicating processing information relating to the first external processing, and a second thumbnail image indicating a state of the radiation image after being transmitted to a transmission destination of the second external processing.

14. The information processing apparatus according to claim 13, wherein the display control unit changes a display form of the second thumbnail image to a display form of the first thumbnail image in a case where processing information relating to the second external processing is received.

15. The information processing apparatus according to claim 13, wherein the display control unit displays, on the first thumbnail image, an icon for identifying the thumbnail image for the radiation image and the first thumbnail image indicating processing information relating to the first external processing.

16. The information processing apparatus according to claim 15, wherein the display control unit displays an external processing apparatus that has executed external processing and processing content of the processing information by changing a display form of the icon.

17. An information processing apparatus comprising:
a determining unit configured to determine one of a plurality of transmission destinations as a transmission destination for executing external processing of a radiation image using processing information relating to the radiation image; and
a transmitting unit configured to transmit the radiation image to the determined transmission destination.

18. A radiation imaging system comprising:
a radiation detector; and
the information processing apparatus according to claim 1 configured to process a radiation image obtained via imaging using the radiation detector.

19. An information processing method comprising:
determining whether to execute second external processing on a radiation image using processing information relating to a first external processing of the radiation image; and
transmitting the radiation image to a transmission destination of the second external processing in a case where it is determined to execute the second external processing.

20. A non-transitory computer-readable storage medium storing a program for causing a computer to execute the method according to claim 19.
